(19) United States
(12) Patent Application Publication
(10) Pub. No.: US 2021/0348102 A1
(43) Pub. Date: Nov. 11, 2021

(54) CELL CULTURE DEVICE FORMING A THREE DIMENSIONAL PERFUSION NETWORK FROM A PATTERNED MATERIAL UPON EXPOSURE TO HYDROGEL

(71) Applicant: SYNO Biotech Inc., Oakville (CA)

(72) Inventor: Boyang Zhang, Oakville (CA)

(21) Appl. No.: 17/268,437

(22) PCT Filed: Aug. 12, 2019
(86) PCT No.: PCT/CA2019/051100

§ 371 Date: Feb. 12, 2021

Related U.S. Application Data
(60) Provisional application No. 62/718,594, filed on Aug. 14, 2018.

(51) Int. Cl. C12M 1/12; C12M 1/00

(52) U.S. Cl. CPC C12M 25/14; C12M 29/10

(57) ABSTRACT

The present invention provides chambers for cell culture that form a three-dimensional perfusion network, comprising a sacrificial material, wherein the patterned portion of the sacrificial material dynamically changes shape three-dimensionally upon exposure to a hydrogel solution. Said chambers for cell culture additionally comprise a first extension portion that extends into a first orifice and anchors the patterned portion of the sacrificial material within the chamber and can partially or fully seal the first orifice from exposure to the hydrogel.

### CROSS-REFERENCE TO RELATED APPLICATIONS

This application claims the benefit of and priority from United States Provisional Patent Application No. 62/718,594 filed on Aug. 14, 2018 which is hereby incorporated by reference in its entirety.

### FIELD

The present application relates to devices and methods that can be applied for biofabrication, in particular, three-dimensional (3D) cellular models.

### BACKGROUND

The increasingly expensive drug development process is one of the major contributors to today's rising healthcare costs. As spending on drug development increases over the past 20 years, the number of drugs approved annually has, in fact, declined. Today, it takes nearly 2.5 billion dollars and 10-12 years on average to develop one clinically applicable drug. Two-thirds of the total costs are spent in clinical trial stages. Hence, late-stage failures can significantly drive up costs and patient risks. Unfortunately, the traditional drug development models of single cell screening often fail to predict drug effects observed at clinical trial stages. To curb the high cost of drug development, the predictive power of pre-clinical screening needs to be improved via more accurate modeling of human physiology to eliminate ineffective drug candidates as early as possible.

Three-dimensional (3D) cellular models offer greater predictivity of gene and protein expression, metabolic function, and physiological and functional readouts than standard two-dimensional (2D) cell culture models. However, achieving high-fidelity 3D tissues remains a major outstanding challenge. Two distinct approaches have emerged over the last several years: organoid technology, spearheaded largely by stem cell biologists; and organ-on-a-chip engineering, led mainly by bioengineers. The two fields use distinct techniques to achieve the same goal of high-fidelity 3D tissue generation. An organoid is a miniaturized and simplified version of an organ produced by the self-assembly of differentiating cells. Organoids possess the advantage of structural sophistication, but are limited by the lack of perfusion and vascularization in vitro, so the self-assembled biological structure cannot be properly accessed as native tissues are in vivo. The organ-on-a-chip approach is based on basic engineering principles, in which a complex system is analyzed by breaking it into pieces and the simplified version of the system is synthesized to fulfill the critical functions of the original system. Perfusion and vascular interfaces can be incorporated into the model to establish a more dynamic micro-environment, but at the expense of oversimplification and tissue fidelity.

What are needed to bridge the gap between organoids and organs-on-a-chip are in vitro models that possess complex perfusable biological structures, such as a 3D vascular-tubular network, that accurately mimic specific tissues, organs, or organ systems.

### SUMMARY

In one aspect, there is provided a chamber for cell culture comprising a sacrificial material and a first orifice, wherein the sacrificial material comprises a patterned portion and a first extension portion and dynamically changes shape three-dimensionally upon exposure to a hydrogel solution, and wherein the first extension portion extends to the first orifice and anchors the patterned portion within the chamber.

In an embodiment of the chamber for cell culture as described herein, the first extension portion extends through the first orifice. The first extension portion may at least partially seal the first orifice upon exposure to the hydrogel solution, thereby anchoring the patterned portion.

In an embodiment of the chamber for cell culture as described herein, the chamber further comprises a second orifice and the sacrificial material further comprises a second extension portion, and wherein the second extension portion extends to the second orifice and optionally anchors the patterned portion within the chamber.

In an embodiment of the chamber for cell culture as described herein, the sacrificial material is alginate, gelatin, Matrigel®, agarose, collagen, polyesters, fibrin, or a combination thereof.

In an embodiment of the chamber for cell culture as described herein, the sacrificial material is alginate.

In an embodiment of the chamber for cell culture as described herein, the size of the cross section of the sacrificial material is from about 100 μm² to about 22,500 μm², or from about 400 μm² to about 10,000 μm².

In an embodiment of the chamber for cell culture as described herein, the patterned portion is in the form of one or more networks. The network may mimic a blood or lymph vessel network, the architecture of an organ or a tissue, or a cavity of an organ or a tissue.

In an embodiment of the chamber for cell culture as described herein, the patterned portion of the sacrificial material is removably attached to the bottom surface of the chamber. The patterned portion may at least partially detach from the bottom surface of the chamber upon exposure to the hydrogel solution.

In another aspect, there is provided a cell culture device comprising a first chamber and a second chamber, wherein the first chamber comprises a sacrificial material and a first orifice, wherein the sacrificial material comprises a patterned portion and a first extension portion and dynamically changes shape three-dimensionally upon exposure to a hydrogel solution, wherein the first extension portion extends to the first orifice and anchors the patterned portion within the first chamber, and wherein the second chamber is in fluid communication with the first chamber via the first orifice.

In an embodiment of the cell culture device as described herein, the first extension portion extends through the first orifice and into the second chamber. The first extension portion may at least partially seal the first orifice upon exposure to the hydrogel solution, thereby anchoring the patterned portion.

In an embodiment of the cell culture device as described herein, the sacrificial material is alginate, gelatin, Matrigel®, agarose, collagen, polyesters, fibrin, or a combination thereof.

In an embodiment of the cell culture device as described herein, the size of the cross section of the sacrificial material is from about 100 μm² to about 22,500 μm².

In another aspect, there is provided a method of constructing a chamber for cell culture, comprising the steps of:
a. assembling a mold comprising a template sheet patterned with a network and a backing sheet;
b. casting a sacrificial material in the mold;
c. solidifying the sacrificial material within the patterned network to form a patterned portion and at least one extension portion;
d. removing the template sheet from the sacrificial material and backing sheet; and
e. assembling a bottomless chamber for cell culture onto the backing sheet such that the patterned portion of the sacrificial material is anchored within the chamber, and the extension portion of the sacrificial material extends to an orifice of the chamber.

In another aspect, there is provided a method of constructing a 3D perfusable network, comprising the steps of:
a. adding a hydrogel solution to the chamber of any one of claims 1 to 12, or the cell culture device of any one of claims 13 to 17, such that the sacrificial material is completely immersed within the hydrogel solution;
b. cross-linking the hydrogel solution; and
c. degrading the sacrificial material.

In another aspect, there is provided a chamber for cell culture comprising:
a. a hydrogel comprising a 3D perfusable network; and
b. an inlet; and
c. optionally, an outlet;

wherein the inlet is a void within the hydrogel through which the network can be perfused, and wherein the inlet is an integral component of the network.

Other aspects and features of the present invention will become apparent to those of ordinary skill in the art upon review of the following description of specific embodiments of the invention in conjunction with the accompanying figures.

### BRIEF DESCRIPTION OF DRAWINGS

FIG. 1A illustrates a method of fabricating a 384-well plate containing 128 independent alginate fiber networks. (1) Fabricating a SU-8 master with an array of various vascular patterns with an inlet channel and an outlet channel with standard soft lithography technique and then molding PDMS against the SU-8 master mold. (2) Punching out wells at the inlet and outlet position of network features on the PDMS mold using a bore with 2 to 2.5 mm diameter. (3) Bonding the no-feature side of the PDMS mold (PDMS mold 1) to a flat silicon wafer by plasma treating both surfaces. (4) Replicating another PDMS mold (PDMS mold 2) against PDMS mold 1 on the silicon wafer. Bonding the PDMS mold 2 against another flat silicon wafer by plasma treating the surfaces. (5) Replicating another PDMS mold (PDMS mold 3) against the PDMS mold 2. (6) Capping the PDMS mold 3 on a polystyrene sheet to form an array of micro-channel networks. (7) Loading the networks with an alginate solution under a low vacuum. (8) Immersing the entire mold in a calcium bath. (9) Removing the calcium bath and air-drying the alginate fibers. (10) Loading a polyethylene glycol-dimethyl ether (PEG-DE) solution into the channels to encase the alginate fibers. (11) Solidifying the PEG-DE. (12) Removing the PDMS mold to leave behind an array of alginate fiber networks encapsulated in PEG-DE on a polystyrene sheet. (13) Assembling the polystyrene sheet onto the base of a bottomless 384-well plate. (14) Washing the wells with distilled water to dissolve away the PEG-DE shell and reveal the alginate networks.

FIG. 1B illustrates the actual products of various steps of the fabrication process shown in FIG. 1A.

FIG. 2A illustrates a 384-well plate containing 128 independent alginate fiber networks encapsulated in PEG-DE in a 384-well plate.

FIG. 2B illustrates a method of fabricating a 384-well plate containing 128 independent 3D perfusable networks. (1) Schematic of three wells of the plate containing the two extension portions and one patterned portion of an alginate network; (2) addition of hydrogel to a chamber containing the patterned portion of an alginate network, with concurrent 3D shape-changing of the alginate; (3) degradation of the alginate network by addition of ethylenediaminetetraacetic acid (EDTA) solution; (4) the resulting device containing a central chamber with a 3D perfusable network in a hydrogel in fluid communication with an inlet and an outlet chamber.

FIG. 2C illustrates perfusion of a formed 3D network in a hydrogel (Collagen I/Matrigel®) with particles (1 μm, green) tagged with fluorescein isothiocyanate (FITC). Dotted lines outline the edges of each well. The arrow shows flow direction. Out-of-focused parts of the network are located outside of the focal plane.

FIG. 2D is a brightfield image of a 3D network coated with endothelial cells.

FIG. 3 illustrates a variety of 3D networks derived from an initial design shown on the left based on organ-specific vascular architecture, and the resulting 3D network perfused with FITC-tagged particles (1 μm, green) and particles (1 μm, red) tagged with tetramethylrhodamine isothiocyanate (TRITC) for visualization on the right. The initial designs are based on organ-specific vascular architecture, namely: (a) convoluted proximal tubules in the kidney; (b) a generic branched vessel; (c) intricately folded glomerulus vessels in the kidney; (d) densely packed vessels in the liver; (e) well-aligned vessels in the muscle; (f) a proximal tubule and the surrounding microvasculature in the kidney; and (g) alveoli and underlying microvasculature in the lung.

FIG. 4A illustrates a plate that includes 128 perfusable networks, each configured with a single inlet and outlet. Designs used in this configuration are used to model: (1) a tubular vessel; (2) a constricted vessel; (3) a convoluted vessel; (4) a generic bifurcating, branched vessel network; (5) a kidney glomerulus vessel; (6) protruded intestinal vessels; (7) liver vessels; and (8) muscle vessels.

FIG. 4B illustrates a network configuration that includes three independently perfused fluid networks, each connected to its own inlet and outlet, that interface at a single well. Designs used in this configuration are used to model: (1) kidney vascular-peritubular networks; (2) pulmonary vascular-alveolar networks; (3) vascular gastrointestinal networks; and (4) vascular-placenta networks.

FIG. 5 illustrates two 3D perfusable networks constructed from alginate patterned using the same initial branched-network mold but immersed in hydrogel formulations of different stiffness to achieve a different final shape. (a) 3D perfusable network formed in hydrogel containing 70% collagen, 10% Matrigel, and 20% PBS (phosphate-buffered saline). (b) 3D perfusable network formed in hydrogel containing 80% Matrigel and 20% PBS.

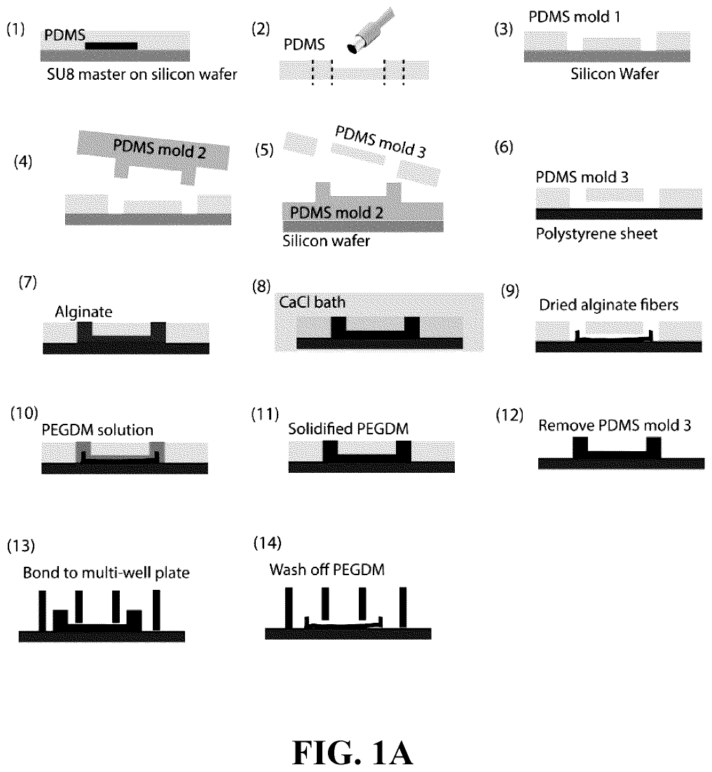

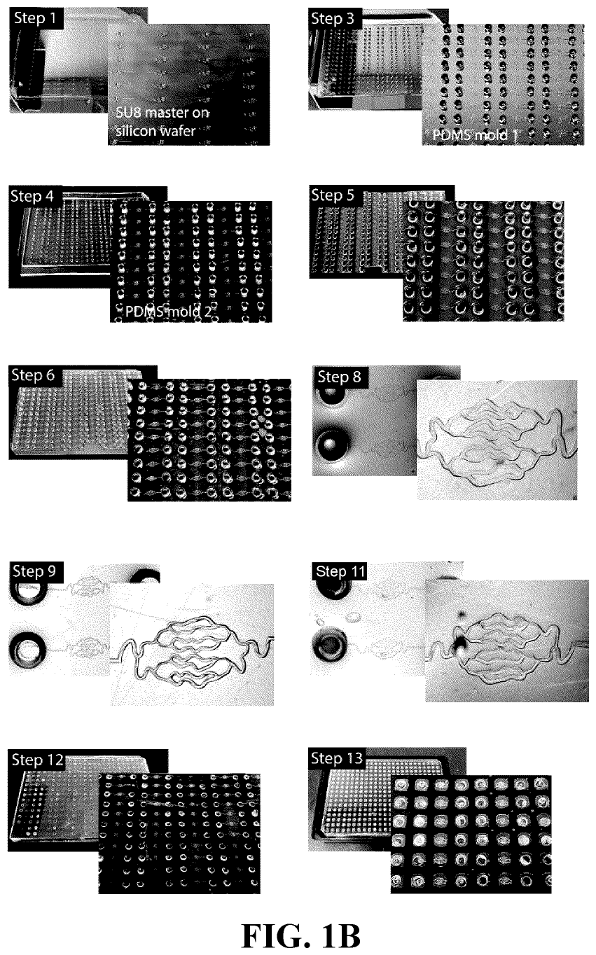

### DETAILED DESCRIPTION

The present inventor has surprisingly discovered that sacrificial materials can be used to carve out 3D perfusable networks that resemble biological structures such as blood vessels or organ-specific perfusable networks in a hydrogel. The 3D perfusable networks can be subsequently populated with various cells to model complex biological structures for biological studies or pharmaceutical drug testing.

The present inventor has further developed devices for 3D cell culture, such as multi-chamber cell culture plates on which a large array (e.g., 40 to 128) of 3D perfusable networks can be readily fabricated, cultured, perfused, and tested in a high-throughput manner. These devices resemble organ-on-a-chip devices in being perfusable to allow access into the internal tissue structure and assessment of biological function, and additionally offer superior structural sophistication and fidelity to biological tissue approaching that seen in stem cell-derived organoids.

Therefore, these devices can serve as a universal platform to model a wide range of biological networks and organ systems.

As used herein, a “perfusable” network is a channel or a series of interconnected channels through which a liquid medium can flow or spread.

As used herein, the term “3D cell culture” means a culture of living cells within a device having three-dimensional structures that mimic the structure, physiology, vasculature, and/or other properties of biological tissues.

In one aspect, there is provided a chamber for cell culture comprising a sacrificial material and a first orifice, wherein the sacrificial material comprises a patterned portion and a first extension portion and is capable of dynamically changing shape three-dimensionally upon exposure to a hydrogel solution, and wherein the first extension portion extends to the first orifice and anchors the patterned portion within the chamber.

As used herein, a “sacrificial material” is a material that that degrades upon exposure to a stimulus. A stimulus that degrades a sacrificial material may include, but is not limited to, a change in temperature, a change in pH, light exposure, addition or removal of a chemical, addition or removal of a biological agent, ultrasound, application of an electromagnetic field, or any combination thereof. When a sacrificial material is embedded or immersed within a different material that is non-responsive to the same stimulus, degradation of a sacrificial material leaves behind a void space (e.g., in the form of a channel) in the different material that is non-responsive to the same stimulus.

Sacrificial materials that may be used in the present invention should have at least one of the following characteristics: (1) flexible; (2) patternable; and (3) compatible with a hydrogel. In the context of the present invention, flexible materials are those capable of bending easily without breaking and readily responding to stimuli (e.g., induced swelling after immersion in water); patternable materials are those capable of being given a regular or intelligible form; and materials compatible with a hydrogel are those that do not chemically react with the hydrogel. In some embodiments, sacrificial materials that may be used in the present invention are 1) flexible; (2) patternable; and (3) compatible with a hydrogel. In some embodiments, sacrificial materials that may be used in the present invention are also nontoxic. In the context of the present invention, “nontoxic” means not substantially interfering with the viability of cells or tissues.

Examples of sacrificial materials that may be used in the present invention include, but are not limited to, alginate, gelatin, Matrigel®, agarose, collagen, polyesters, and fibrin.

In some embodiments, the sacrificial material is alginate. Alginate, also known as alginic acid or algin, is a polysaccharide naturally existing in brown algae. Alginate can be rapidly cross-linked in the presence of calcium and then rapidly degraded in the absence of calcium. Therefore, withdrawal of calcium (e.g., as a result of addition of a chelating agent, such as ethylenediaminetetraacetic acid (EDTA)) can serve as a stimulus that degrades alginate.

In some embodiments, the sacrificial material is Matrigel. Matrigel is the trade name for a gelatinous protein mixture secreted by Engelbreth-Holm-Swarm mouse sarcoma cells. Matrigel solidifies to form a gel when incubated at 37° C. Matrigel can be degraded by dispase.

In some embodiments, the sacrificial material is agarose. Agarose is a purified linear galactan hydrocolloid, generally extracted from agar-bearing marine algae. Agarose gels and melts at different temperatures, which vary depending on the type of agarose. Therefore, heating can serve as a stimulus that degrades agarose.

In some embodiments, the sacrificial material is collagen. Collagen is the main structural protein in the extracellular space in the various connective tissues of animals. Collagen fibrils self-assemble when a solution of collagen is heated. Collagen gels can be degraded by collagenases.

In some embodiments, the sacrificial material is a polyester. A polyester is a polymer that contains the ester functional group in its main chain. Polyesters undergo degradation by hydrolysis under acidic or basic conditions. Therefore, a change in pH can serve as a stimulus that degrades polyesters.

In some embodiments, the sacrificial material is fibrin. Fibrin is a natural protein formed during wound coagulation. Selective cleavage of the dimeric glycoprotein fibrinogen by the serine protease thrombin results in the formation of fibrin molecules that crosslink through disulfide bond formation. Fibrin can be degraded by proteases such as nattokinase.

Further examples of sacrificial materials that may be used in the present invention include, but are not limited to, polysaccharides, hyaluronic acid, xanthan gums, natural gum, agar, carrageenan, fucoidan, furcellaran, laminaran, hypnea, eucheuma, gum arabic, gum ghatti, gum karaya, gum tragacanth, locust beam gum, arabinogalactan, pectin, amylopectin, and ribo- or deoxyribonucleic acids.

In some embodiments, sacrificial materials that may be used in the present invention may have a cross section size of about 100 μm² to about 1,000,000 μm². In some embodiments, the size of the cross section of the sacrificial material is from about 100 μm² to about 640,000 μm². In some embodiments, the size of the cross section of the sacrificial material is from about 100 μm² to about 360,000 μm². In some embodiments, the size of the cross section of the sacrificial material is from about 100 μm² to about 160,000 μm². In some embodiments, the size of the cross section of the sacrificial material is from about 100 μm² to about 40,000 μm². In some embodiments, the size of the cross section of the sacrificial material is from about 100 μm² to about 22,500 μm². In some embodiments, the size of the cross section of the sacrificial material is from about 400 μm² to about 10,000 μm². In some embodiments, the size of the cross section of the sacrificial material is from about 400 μm² to about 6,400 μm². In some embodiments, the size of the cross section of the sacrificial material is from about 400 μm² to about 3,600 μm². In some embodiments, the size of the cross section of the sacrificial material is from about 400 μm² to about 1,600 μm².

In some embodiments, the size of the cross section of the sacrificial material is about 100 μm². In some embodiments, the size of the cross section of the sacrificial material is about 400 μm². In some embodiments, the size of the cross section of the sacrificial material is about 900 μm². In some embodiments, the size of the cross section of the sacrificial material is about 1,600 μm². In some embodiments, the size of the cross section of the sacrificial material is about 2,500 μm². In some embodiments, the size of the cross section of the sacrificial material is about 3,600 μm². In some embodiments, the size of the cross section of the sacrificial material is about 4,900 μm². In some embodiments, the size of the cross section of the sacrificial material is about 6,400 μm². In some embodiments, the size of the cross section of the sacrificial material is about 8,100 μm². In some embodiments, the size of the cross section of the sacrificial material is about 10,000 μm². In some embodiments, the size of the cross section of the sacrificial material is about 12,100 μm². In some embodiments, the size of the cross section of the sacrificial material is about 14,400 μm². In some embodiments, the size of the cross section of the sacrificial material is about 16,900 μm². In some embodiments, the size of the cross section of the sacrificial material is about 19,600 μm².

In a chamber for cell culture provided herein, the sacrificial material comprises a patterned portion and a first extension portion. The patterned portion has a regular or intelligible form. For example, the patterned portion may be in the form of one or more networks, each of which may mimic a blood or lymph vessel network, the architecture of an organ or a tissue, or a cavity of an organ or a tissue (e.g., pulmonary alveoli). In some embodiments, the patterned portion of the sacrificial material is prepared using microfabrication techniques. As used herein, the term “microfabrication” means fabrication on a nanometer or micrometer level, including nanofabrication. Microfabrication techniques may be additive or subtractive in nature. Microfabrication techniques include, but are not limited to, photolithography, soft lithography, micromolding (e.g., injection molding, hot embossing, and casting), 3D printing (e.g., inkjet 3D printing, stereolithography, two-photon polymerisation, and extrusion printing), micromilling, and bonding techniques.

The first extension portion of the sacrificial material is a portion of the sacrificial material that is configured to extend to the first orifice of the chamber. In the context of the present invention, a structure extends to an orifice when the structure reaches the orifice, or extends into the orifice but does not penetrate the orifice completely, or extends through the orifice (i.e., the structure penetrates the orifice completely and reaches outside the orifice).

In some embodiments, in a chamber for cell culture provided herein, the size of the cross section of the first orifice is no more than about 1000 times, no more than about 900 times, no more than about 800 times, no more than about 700 times, no more than about 600 times, no more than about 500 times, no more than about 400 times, no more than about 300 times, no more than about 200 times, no more than about 100 times, no more than about 90 times, no more than about 80 times, no more than about 70 times, no more than about 60 times, no more than about 50 times, no more than about 40 times, no more than about 30 times, no more than about 20 times, no more than about 15 times, no more than about 10 times, no more than about 9 times, no more than about 8 times, no more than about 7 times, no more than about 6 times, no more than about 5 times, no more than about 4 times, no more than about 3 times, or no more than about 2 times, larger than the size of the cross section of the first extension portion of the sacrificial material.

The first extension portion of the sacrificial material also serves to anchor the patterned portion of the sacrificial material within the chamber. In the context of the present invention, the patterned portion of the sacrificial material is anchored within the chamber when the patterned portion is not freely floating within the chamber when a hydrogel solution is added to the chamber. In some embodiments, the first extension portion swells upon exposure to a hydrogel solution and partially or completely seals the first orifice, thereby anchoring the patterned portion. As used herein, swelling of a material refers to an increase in size of the material caused by an accumulation or absorption of a fluid such as water. In some embodiments, the first extension portion is removably or permanently attached to an exterior surface, thereby anchoring the patterned portion in the absence of a hydrogel.

Without being limited by theory, it is believed that anchoring of the patterned portion permits the patterned portion to dynamically changing shape three-dimensionally in the chamber upon exposure to a hydrogel solution. As used herein, the shape of a material refers to its external physical form in three dimensions. In the context of the present invention, changing shape means altering the external form of an object in any way other than an isotropic scaling (i.e., a mere increase or decrease in size of an object is not shape changing), and dynamically changing shape refers to changing shape in a manner characterized by constant change as a function of time. The shape-changing behavior of the patterned portion upon exposure to a hydrogel solution has a degree of stochasticity in that the exact positioning and shape of the sacrificial material network in the 3D space is not predetermined, which is desirable as natural and bio-inspired stochasticity enables high phenotype fidelity and physiologically relevant complexity. At the same time, distinct organizations of complex networks originating from various organs or tissues or even various parts of an organ can be captured, as the pattern of the patterned portion can pre-define cross section size, density and shape of a 3D network as well as the frequency and location of the branches.

Without being limited by theory, it is further believed that the interaction between the hydrogel and the sacrificial material plays a role in determining the final shape of the sacrificial material network in the 3D space.

In some embodiments, the patterned portion of the sacrificial material is removably attached to the bottom surface of the chamber, and at least partially detaches from the bottom surface of the chamber upon exposure to the hydrogel solution, thereby allowing the patterned portion to dynamically changing shape three-dimensionally in the chamber.

As used herein, a “hydrogel” is a hydrophilic polymeric network cross-linked in some fashion to produce a structure that can contain a significant amount of water. Suitable hydrogel polymers for the present invention may include, but are not limited to, polyvinyl alcohol, sodium polyacrylate, polyacrylamide, polyethylene glycol, polylactic acid, polyglycolic acid, agarose, methylcellulose, hyaluronan, collagen (e.g., Matrigel® and HuBiogel®), fibrin, alginate, polypeptides, other synthetic or naturally derived polymers or copolymers with an abundance of hydrophilic groups, and any combination thereof. In the context of the present invention, a hydrogel polymer cannot be the same as the sacrificial material of a chamber for cell culture provided herein. In some embodiments, the hydrogel polymer is suitable for use in cell culture. In some embodiments, the hydrogel polymer is collagen, Matrigel®, or a mixture thereof.

In the context of the present invention, a hydrogel may be formed by cross-linking a hydrogel solution comprising a hydrogel polymer and a solvent. The cross-linking may occur as a result of a change in temperature, a change in pH, light exposure, addition or removal of a chemical, addition or removal of a biological agent, ultrasound, application of an electromagnetic field, or any combination thereof. Suitable solvents for hydrogel polymers may include, but are not limited to, water, aqueous buffers, and cell culture media.

In some embodiments, a hydrogel solution that may be used in the present invention contains at least 50% water by mass. In some embodiments, the hydrogel solution contains at least 90% water by mass. In some embodiments, the hydrogel solution contains at least 95% water by mass. In some embodiments, the hydrogel solution contains at least 98% water by mass. In some embodiments, the hydrogel solution contains at least 99% water by mass.

In some embodiments, the temperature to be used for hydrogel cross-linking is from about 4° C. to about 45° C. In some embodiments, the temperature to be used for hydrogel cross-linking is about 25° C., 30° C., 37° C. or 42° C.

In some embodiments, a chamber for cell culture provided herein may comprise more than one orifice and the sacrificial material may comprise more than one extension portion. For example, the sacrificial material may comprise a second extension portion, wherein the second extension portion extends to a second orifice of the chamber. In some embodiments, the second extension portion also anchors the patterned portion within the chamber.

In some embodiments, the size of the cross section of the second orifice is no more than about 1000 times, no more than about 900 times, no more than about 800 times, no more than about 700 times, no more than about 600 times, no more than about 500 times, no more than about 400 times, no more than about 300 times, no more than about 200 times, no more than about 100 times, no more than about 90 times, no more than about 80 times, no more than about 70 times, no more than about 60 times, no more than about 50 times, no more than about 40 times, no more than about 30 times, no more than about 20 times, no more than about 15 times, no more than about 10 times, no more than about 9 times, no more than about 8 times, no more than about 7 times, no more than about 6 times, no more than about 5 times, no more than about 4 times, no more than about 3 times, or no more than about 2 times, larger than the size of the cross section of the second extension portion of the sacrificial material.

In some embodiments, the second extension portion swells upon exposure to a hydrogel solution and partially or completely seals the second orifice, thereby anchoring the patterned portion. In some embodiments, the second extension portion is removably or permanently attached to an exterior surface, thereby anchoring the patterned portion in the absence of a hydrogel.

In some embodiments, a chamber for cell culture provided herein may comprise a plurality of patterned portions, each of which may be connected to one or two extension portions. Each patterned portion may be designed to capture the specific characteristics of a specific tubular network found in different organs or tissues. For example, the tubular network may be a straight tubular vessel, a convoluted vessel that decouples the biological effects of vessel curvature, a constricted vessel that can model vascular diseases, a generic bifurcation branched vessel network that provides a generic vascular bed, or a network that captures the specific architecture of an organ or a tissue. The plurality of patterned portions together may enable the tubular networks to form an intercommunicating system that can carry out physiological functions.

In another aspect, there is provided a cell culture device comprising at least one chamber for cell culture provided herein. In some embodiments, a cell culture device provided herein comprises a second chamber, wherein the second chamber is in fluid communication with the first chamber via the first orifice. In some embodiments, the first extension portion of the sacrificial material extends through the first orifice and into the second chamber.

In some embodiments, when a chamber for cell culture provided herein comprises a first orifice and a second orifice and the sacrificial material comprises a first extension portion and a second extension portion, a cell culture device comprising the chamber for cell culture provided herein comprises a second chamber and a third chamber, wherein the second chamber is in fluid communication with the first chamber via the first orifice and the third chamber is in fluid communication with the first chamber via the second orifice. In some embodiments, the first extension portion of the sacrificial material extends through the first orifice and into the second chamber and the second extension portion of the sacrificial material extends through the second orifice and into the third chamber.

In some embodiments, the cell culture device is a multi-chamber cell culture plate that contains 3, 4, 6, 8, 9, 12, 24, 48, 96, 384, or 1536 chambers. In some embodiments, the cell culture device is a flask or roller bottle.

In some embodiments, the cell culture device is for the culture of eukaryotic cells. In some embodiments the cell culture device is for the culture of mammalian cells including, but not limited to, undifferentiated cell types (e.g., induced pluripotent stem cells, embryonic stem cells, and mesenchymal stem cells), as well as differentiated cell types.

In some embodiments, differentiated cell types to be cultured include neurons, astrocytes, oligodendrocytes, microglia, hepatocytes, cardiomyocytes, muscle cells, kidney cells, endothelial cells, epithelial cells, alveolar cells, cartilage cells, fibroblasts, skin cells, bone marrow cells, T-cells, lymphocytes, macrophages, or any combination thereof.

In another aspect, there is provided a method of constructing a chamber for cell culture, comprising the steps of:
a. assembling a mold comprising a template sheet patterned with a network and a backing sheet;
b. casting a sacrificial material in the mold;
c. solidifying the sacrificial material within the patterned network to form a patterned portion and at least one extension portion;
d. removing the template sheet from the sacrificial material and backing sheet; and
e. assembling a bottomless chamber for cell culture onto the backing sheet such that the patterned portion of the sacrificial material is anchored within the chamber, and the extension portion of the sacrificial material extends to an orifice of the chamber.

In some embodiments, the mold comprises a template sheet patterned with recessed regions in contact with a backing sheet to create a patterned network within the mold. The template sheet is typically made of an elastomer such as polydimethylsiloxane (PDMS), a polyurethane, a polyimide, or a cross-linked phenol-formaldehyde polymer, and can be fabricated using microfabrication techniques. In some embodiments, the template sheet may be reused after being removed from the solidified sacrificial material and backing sheet. The backing sheet is typically made of a biologically inert polymer such as polystyrene, polypropylene, polycarbonate or cyclic olefin copolymer.

In some embodiments, casting the sacrificial material may involve filling the patterned network of the mold with a solution of the sacrificial material or its constituent monomers. The sacrificial material may be solidified by curing or evaporating the solvent, thereby obtaining negative transfer of the mold. In some embodiments, the sacrificial material is dried to complete the solidification process.

In some embodiments, the sacrificial material is alginate, which is cured by immersing the mold filled with the alginate solution in a calcium bath.

In some embodiments, the sacrificial material is Matrigel, which is cured by incubating the mold at 37° C.

In some embodiments, the sacrificial material is agarose, which is cured by incubating the mold at the gel point of the agarose solution.

In some embodiments, the sacrificial material is collagen, which is cured by incubating the mold at 37° C.

In some embodiments, the sacrificial material is a polyester, which is cast as a solution of monomers, which is cured by ultraviolet light or heat or solidified by passive solvent evaporation.

In some embodiments, the sacrificial material is fibrin, which is cast as a solution of fibrinogen, which is cured by addition of thrombin.

In some embodiments, the chamber for cell culture is assembled by bonding the bottomless chamber onto the backing sheet. In some embodiments, the bonding is done by gluing the bottomless chamber onto the backing sheet. The glue used may be a nontoxic polyurethane glue. When the bonding is done by gluing, the sacrificial material may be protected during the assembly step by being encapsulated inside an inert water-soluble polymer such as PEG-dimethyl ether, which can be removed after the assembly step by washing with the chamber with water. Encapsulating the sacrificial material can leave behind an orifice to receive the extension portion once the water-soluble polymer is dissolved, thus avoiding the need to create an orifice on a wall of the bottomless chamber before the assembly step.

In some embodiments, a micro-groove is patterned (e.g., using micro-drilling or hot embossing) on the bottom edge of the bottomless chamber, such that it aligns with and/or encases the extension portion of the sacrificial material during the assembly step to form an orifice. The assembly step may be then performed using an ultrasonic welder.

In another aspect, there is provided a method of constructing a 3D perfusable network, comprising the steps of:
a. adding a hydrogel solution to a chamber for cell culture or a cell culture device provided herein such that the sacrificial material is completely immersed within the hydrogel solution;
b. cross-linking the hydrogel solution; and
c. degrading the sacrificial material.

The shape of the lumen in the channels in a 3D perfusable network constructed in accordance with this method is not limited in any particular manner and may be square, rectangular, circular, oval, oblong, triangular, or any combination of shapes. The height and width of the lumen also may vary in any suitable manner. The other dimensions of the channels, such as their length and volume, also may vary in any suitable manner.

In some embodiments, the surface of a channel in a 3D perfusable network constructed in accordance with this method may be modified with any suitable surface treatments, including chemical modifications (such as, for example, ligands, charged substances, binding agents, growth factors, antibiotics, antifungal agents), and physical modifications (such as, for example, spikes, curved portions, folds, pores, uneven portions, or various shapes and topographies), or any combination thereof, which may facilitate a cell culture process.

In some embodiments, the sacrificial material is alginate, which is degraded by adding ethylenediaminetetraacetic acid (EDTA) to the chamber or device containing the alginate.

In some embodiments, the sacrificial material is Matrigel, which is degraded by adding dispase to the chamber or device containing Matrigel.

In some embodiments, the sacrificial material is agarose, which is degraded by heating the chamber or device to the melting temperature of the agarose.

In some embodiments, the sacrificial material is collagen, which is degraded by adding a collagenase to the chamber or device containing the collagen.

In some embodiments, the sacrificial material is a polyester, which is degraded by adding an acid or base to the chamber or device containing the polyester.

In some embodiments, the sacrificial material is fibrin, which is degraded by adding a protease such as nattokinase to the chamber or device containing the fibrin.

In a chamber and device provided herein, at least one extension portion of the sacrificial material anchors the patterned portion of the sacrificial material such that the patterned portion does not freely float within the chamber or chambers when a hydrogel solution is added. At least one extension portion of the sacrificial material extends to, into, or through an orifice in the chamber such that, after the hydrogel solution is added and cross-linked and the sacrificial material is degraded, the orifice serves as an inlet or outlet through which the constructed 3D perfusable network can be perfused.

In some embodiments, a constructed 3D perfusable network may be perfused with water or an aqueous solution. In some embodiments, a constructed 3D perfusable network may be perfused with a liquid medium containing cells. In some embodiments, a constructed 3D perfusable network may physically support the attachment of cells and/or molecules.

In some embodiments, a plurality of 3D perfusable networks may be constructed according to methods provided herein, at least two of which can be independently perfused.

When a cell culture device comprises a plurality of chambers provided herein, a plurality of 3D perfusable networks can be constructed after addition and cross-linking of a hydrogel solution and degradation of the sacrificial material. The plurality of 3D perfusable networks may vary in the exact 3D shape which is stochastically determined, while sharing the same general architecture predetermined by the pattern of the patterned portion. By allowing such a variety of 3D perfusable networks to be incorporated on the same plate, the invention enables the stochasticity of biological vascular networks to be modelled on a single 3D cell culture plate.

In another aspect, there is provided a method of 3D cell culturing, comprising the steps of:
a. adding a hydrogel solution to a chamber for cell culture or a cell culture device provided herein such that the sacrificial material is completely immersed within the hydrogel solution;
b. cross-linking the hydrogel solution;
c. degrading the sacrificial material such that at least one 3D perfusable network is formed; and
d. perfusing the 3D perfusable network with a liquid medium containing cells.

In another aspect, there is provided a kit comprising a chamber for cell culture or a cell culture device provided herein, and a hydrogel solution.

In another aspect, there is provided a chamber for cell culture comprising:
a. a hydrogel comprising a 3D perfusable network; and
b. an inlet; and
c. optionally, an outlet;

wherein the inlet is a void within the hydrogel through which the network can be perfused, and wherein the inlet is an integral component of the network.

As used herein, “integral” means that the inlet is fabricated in the same manner and at the same time as the 3D perfusable network. For example, if the 3D perfusable network and the inlet are simultaneously fabricated by degrading an alginate network within the hydrogel by addition of EDTA, then the inlet is an integral component of the network. In the context of the present invention, an inlet that is fabricated by perforating the hydrogel in a step subsequent to fabrication of the perfusable network is not an integral component of the network.

In some embodiments, the outlet is a void within the hydrogel through which the network can be perfused, and wherein the inlet is an integral component of the network.

Chambers and devices provided herein may be used for 3D cell culture that mimics the structure, physiology, vasculature, and other properties of biological tissues. Biological tissues may include, but are not limited to, cardiac, hepatic, neural, vascular, kidney, gastrointestinal, placental, and muscle tissues. Methods and devices provided herein are suitable for high-throughput experimentation, and may be used in a variety of applications that include fundamental biological and medical research, drug discovery, medical diagnostics, and tissue engineering. Examples of such applications include: (a) testing of the efficacy and safety (including toxicity) of pharmacologic agents; (b) defining of pharmacokinetics and/or pharmacodynamics of pharmacologic agents; (c) characterizing the properties and therapeutic effects of pharmacologic agents, including their ability to penetrate an endothelial cell barrier; (d) screening of new pharmacologic agents; (e) delivery of pharmacologic agents; (f) modelling barrier function within a tissue or organ; (g) modelling functionality of the parenchymal tissue of an organ; (h) modelling the systematic interaction between various tissues and organs of the body; (i) tissue repair and/or treatment in regenerative medicine; (j) histology; (k) personalized medicine; and (l) bioseparations. Pharmacologic agents may include, but are not limited to, small-molecule drugs, biologics (e.g., proteins, peptides, antibodies, lipids, and polysaccharides), nucleic acid-based agents, supplements, diagnostic agents, and immune modulators.

Methods and devices provided herein can be used to engineer a broad range of tissue types with high biological fidelity, which may enable high-throughput screening of multi-organ interactions on a single universal platform. Such “clinical-trials-on-a-chip” could collect large amounts of data from an array of independent biological systems that may be useful for uncover subtle biological responses that offer important biological insights, for example, capturing unexpected drug toxicities in advance of late-stage clinical trials in which a large number of human participants are exposed.

### EMBODIMENTS

Particular embodiments of the invention include, without limitation, the following:
1. A chamber for cell culture comprising a sacrificial material and a first orifice, wherein the sacrificial material comprises a patterned portion and a first extension portion and dynamically changes shape three-dimensionally upon exposure to a hydrogel solution, and wherein the first extension portion extends to the first orifice and anchors the patterned portion within the chamber.
2. The chamber of embodiment 1, wherein the size of the cross section of the first orifice is no more than 100 times larger than the size of the cross section of the first extension portion.
3. The chamber of embodiment 2, wherein the size of the cross section of the first orifice is no more than 10 times larger than the size of the cross section of the first extension portion.
4. The chamber of any one of embodiments 1 to 3, wherein the first extension portion extends into the first orifice.
5. The chamber of any one of embodiments 1 to 4, wherein the first extension portion extends through the first orifice.
6. The chamber of embodiment 4 or 5, wherein the first extension portion at least partially seals the first orifice upon exposure to the hydrogel solution, thereby anchoring the patterned portion.
7. The chamber of any one of embodiments 1 to 6, wherein the chamber further comprises a second orifice and the sacrificial material further comprises a second extension portion, and wherein the second extension portion extends to the second orifice and optionally anchors the patterned portion within the chamber.
8. The chamber of embodiment 7, wherein the size of the cross section of the second orifice is no more than 100 times larger than the size of the cross section of the second extension portion.
9. The chamber of embodiment 8, wherein the size of the cross section of the second orifice is no more than 10 times larger than the size of the cross section of the second extension portion.
10. The chamber of any one of embodiments 7 to 9, wherein the second extension portion extends into the second orifice.
11. The chamber of any one of embodiments 7 to 10, wherein the second extension portion extends through the second orifice.
12. The chamber of embodiment 10 or 11, wherein the second extension portion at least partially seals the second orifice upon exposure to the hydrogel solution, thereby anchoring the patterned portion.
13. The chamber of any one of embodiments 1 to 12, wherein the sacrificial material is alginate, gelatin, Matrigel®, agarose, collagen, polyesters, fibrin, or a combination thereof.
14. The chamber of any one of embodiments 1 to 13, wherein the sacrificial material is alginate.
15. The chamber of any one of embodiments 1 to 14, wherein the size of the cross section of the sacrificial material is from about 100 μm² to about 22,500 μm².
16. The chamber of any one of embodiments 1 to 15, wherein the size of the cross section of the sacrificial material is from about 400 μm² to about 10,000 μm².
17. The chamber of any one of embodiments 1 to 16, wherein the patterned portion is in the form of one or more networks.
18. The chamber of embodiment 17, wherein the network mimics a blood or lymph vessel network, the architecture of an organ or a tissue, or a cavity of an organ or a tissue.
19. The chamber of any one of embodiments 1 to 18, wherein the patterned portion of the sacrificial material is removably attached to the bottom surface of the chamber.
20. The chamber of embodiment 19, wherein the patterned portion at least partially detaches from the bottom surface of the chamber upon exposure to the hydrogel solution.
21. A cell culture device comprising a first chamber and a second chamber, wherein the first chamber comprises a sacrificial material and a first orifice, wherein the sacrificial material comprises a patterned portion and a first extension portion and dynamically changes shape three-dimensionally upon exposure to a hydrogel solution, wherein the first extension portion extends to the first orifice and anchors the patterned portion within the first chamber, and wherein the second chamber is in fluid communication with the first chamber via the first orifice.
22. The cell culture device of embodiment 21, wherein the size of the cross section of the first orifice is no more than 100 times larger than the size of the cross section of the first extension portion.
23. The cell culture device of embodiment 22, wherein the size of the cross section of the first orifice is no more than 10 times larger than the size of the cross section of the first extension portion.
24. The cell culture device of any one of embodiments 21 to 23, wherein the first extension portion extends into the first orifice.
25. The cell culture device of any one of embodiments 21 to 24, wherein the first extension portion extends through the first orifice and into the second chamber.
26. The cell culture device of embodiment 24 or 25, wherein the first extension portion at least partially seals the first orifice upon exposure to the hydrogel solution, thereby anchoring the patterned portion.
27. The cell culture device of any one of embodiments 21 to 26, wherein the first chamber further comprises a second orifice and the sacrificial material further comprises a second extension portion, and wherein the second extension portion extends to the second orifice and optionally anchors the patterned portion within the chamber.
28. The cell culture device of embodiment 27, wherein the size of the cross section of the second orifice is no more than 100 times larger than the size of the cross section of the second extension portion.
29. The cell culture device of embodiment 28, wherein the size of the cross section of the second orifice is no more than 10 times larger than the size of the cross section of the second extension portion.
30. The cell culture device of any one of embodiments 27 to 29, wherein the second extension portion extends into the second orifice.
31. The cell culture device of any one of embodiments 27 to 30, wherein the second extension portion extends through the second orifice.
32. The cell culture device of embodiment 30 or 31, wherein the second extension portion at least partially seals the second orifice upon exposure to the hydrogel solution, thereby anchoring the patterned portion.
33. The cell culture device of any one of embodiments 27 to 32, wherein the cell culture device further comprises a third chamber, and wherein the third chamber is in fluid communication with the first chamber via the second orifice.
34. The cell culture device of any one of embodiments 21 to 33, wherein the sacrificial material is alginate, gelatin, Matrigel®, agarose, collagen, polyesters, fibrin, or a combination thereof.
35. The cell culture device of any one of embodiments 21 to 34, wherein the sacrificial material is alginate.
36. The cell culture device of any one of embodiments 21 to 35, wherein the size of the cross section of the sacrificial material is from about 100 μm² to about 22,500 μm².
37. The cell culture device of any one of embodiments 21 to 36, wherein the size of the cross section of the sacrificial material is from about 400 μm² to about 10,000 μm².
38. The cell culture device of any one of embodiments 21 to 37, wherein the patterned portion is in the form of one or more networks.
39. The cell culture device of embodiment 38, wherein the network mimics a blood or lymph vessel network, the architecture of an organ or a tissue, or a cavity of an organ or a tissue.
40. The cell culture device of any one of embodiments 21 to 39, wherein the patterned portion of the sacrificial material is removably attached to the bottom surface of the first chamber.
41. The cell culture device of embodiment 40, wherein the patterned portion at least partially detaches from the bottom surface of the first chamber upon exposure to the hydrogel solution.
42. The cell culture device of any one of embodiments 21 to 41, which is a multi-chamber cell culture plate.
43. A method of constructing a chamber for cell culture, comprising the steps of:
   a. assembling a mold comprising a template sheet patterned with a network and a backing sheet;
   b. casting a sacrificial material in the mold;
   c. solidifying the sacrificial material within the patterned network to form a patterned portion and at least one extension portion;
   d. removing the template sheet from the sacrificial material and backing sheet; and
   e. assembling a bottomless chamber for cell culture onto the backing sheet such that the patterned portion of the sacrificial material is anchored within the chamber, and the extension portion of the sacrificial material extends to an orifice of the chamber.
44. The method of embodiment 43, wherein the size of the cross section of the orifice is no more than 100 times larger than the size of the cross section of the extension portion.
45. The method of embodiment 44, wherein the size of the cross section of the orifice is no more than 10 times larger than the size of the cross section of the extension portion.
46. The method of any one of embodiments 43 to 45, wherein the extension portion extends into the orifice.
47. The method of any one of embodiments 43 to 46, wherein the extension portion extends through the orifice.
48. The method of embodiment 46 or 47, wherein the extension portion at least partially seals the orifice upon exposure to a hydrogel solution, thereby anchoring the patterned portion.
49. The method of any one of embodiments 43 to 48, wherein the sacrificial material is alginate, gelatin, Matrigel®, agarose, collagen, polyesters, fibrin, or a combination thereof.
50. The method of any one of embodiments 43 to 49, wherein the sacrificial material is alginate.
51. The method of any one of embodiments 43 to 50, wherein the size of the cross section of the sacrificial material is from about 100 μm² to about 22,500 μm².
52. The method of any one of embodiments 43 to 51, wherein the size of the cross section of the sacrificial material is from about 400 μm² to about 10,000 μm².
53. The method of any one of embodiments 43 to 52, wherein the patterned portion is in the form of one or more networks.
54. The method of embodiment 53, wherein the network mimics a blood or lymph vessel network, the architecture of an organ or a tissue, or a cavity of an organ or a tissue.
55. The method of any one of embodiments 43 to 54, wherein the patterned portion of the sacrificial material is removably attached to the backing sheet.
56. The method of embodiment 55, wherein the patterned portion at least partially detaches from the backing sheet upon exposure to a hydrogel solution.
57. A kit comprising the chamber of any one of embodiments 1 to 20 or the cell culture device of any one of embodiments 21 to 42, and a hydrogel solution.
58. The chamber of any one of embodiments 1 to 20, or the cell culture device of any one of embodiments 21 to 42, or the method of any one of embodiments 43 to 56, or the kit of embodiment 57, wherein the hydrogel solution comprises a hydrogel polymer selected from polyvinyl alcohol, sodium polyacrylate, polyacrylamide, polyethylene glycol, polylactic acid, polyglycolic acid, agarose, methylcellulose, hyaluronan, collagen (e.g., Matrigel® and HuBiogel®), fibrin, alginate, polypeptides, other synthetic or naturally derived polymers or copolymers with an abundance of hydrophilic groups, and any combination thereof.
59. The chamber of any one of embodiments 1 to 20, or the cell culture device of any one of embodiments 21 to 42, or the method of any one of embodiments 43 to 56, or the kit of embodiment 57, wherein the hydrogel solution comprises a hydrogel polymer that is collagen, Matrigel®, or a mixture thereof.
60. A method of constructing a 3D perfusable network, comprising the steps of:
   a. adding a hydrogel solution to the chamber of any one of embodiments 1 to 20, or the cell culture device of any one of embodiments 21 to 42, such that the sacrificial material is completely immersed within the hydrogel solution;
   b. cross-linking the hydrogel solution; and
   c. degrading the sacrificial material.
61. A method of 3D cell culturing, comprising the steps of:
   a. adding a hydrogel solution to a chamber for cell culture or a cell culture device provided herein such that the sacrificial material is completely immersed within the hydrogel solution;
   b. cross-linking the hydrogel solution;
   c. degrading the sacrificial material such that at least one 3D perfusable network is formed; and
   d. perfusing the 3D perfusable network with a liquid medium containing cells.
62. The method of embodiment 60 or 61, wherein the 3D perfusable network is a 3D tubular network.
63. The method of any one of embodiments 60 to 62, wherein the hydrogel solution comprises a hydrogel polymer selected from polyvinyl alcohol, sodium polyacrylate, polyacrylamide, polyethylene glycol, polylactic acid, polyglycolic acid, agarose, methylcellulose, hyaluronan, collagen (e.g., Matrigel® and HuBiogel®), fibrin, alginate, polypeptides, other synthetic or naturally derived polymers or copolymers with an abundance of hydrophilic groups, and any combination thereof.
64. The method of any one of embodiments 60 to 62, wherein the hydrogel solution comprises a hydrogel polymer that is collagen, Matrigel®, or a mixture thereof.
65. A chamber for cell culture comprising:
   a. a hydrogel comprising a 3D perfusable network; and
   b. an inlet; and
   c. optionally, an outlet;

wherein the inlet is a void within the hydrogel through which the network can be perfused, and wherein the inlet is an integral component of the network.
66. The chamber of embodiment 65, wherein the hydrogel comprises a hydrogel polymer selected from polyvinyl alcohol, sodium polyacrylate, polyacrylamide, polyethylene glycol, polylactic acid, polyglycolic acid, agarose, methylcellulose, hyaluronan, collagen (e.g., Matrigel® and HuBiogel®), fibrin, alginate, polypeptides, other synthetic or naturally derived polymers or copolymers with an abundance of hydrophilic groups, and any combination thereof.
67. The chamber of embodiment 65, wherein the hydrogel comprises a hydrogel polymer that is collagen, Matrigel®, or a mixture thereof.
68. The chamber of any one of embodiments 65 to 67, wherein the size of the cross section of a channel of the 3D perfusable network is from about 100 μm² to about 22,500 μm².
69. The chamber of any one of embodiments 65 to 68, wherein the size of the cross section of a channel of the 3D perfusable network is from about 400 μm² to about 10,000 μm².
70. The chamber of any one of embodiments 65 to 69, wherein the 3D perfusable network comprises one or more tubular networks.
71. The chamber of embodiment 70, wherein the tubular network mimics a blood or lymph vessel network, the architecture of an organ or a tissue, or a cavity of an organ or a tissue.

### EXAMPLES

### Example 1: Patterning of a Branched Network of Alginate Fibers with Diameters Ranging from 20 to 100 μm

First, using standard photolithography, a polydimethylsiloxane (PDMS) mold was fabricated with various vascular patterns connected to an inlet and outlet well. The mold was then capped onto a polystyrene sheet to form an array of micro-channel networks. The networks were loaded with 3 wt % alginate solution (Sigma A2158) under a low vacuum (0.04 mPa). Next, the entire mold was immersed in a calcium bath (1 mM calcium chloride), where calcium ions gradually diffused from the inlet and outlet wells into the alginate solution within the networks and crosslinked the alginate overnight. With this approach, 128 independent alginate fiber networks (diameter<100 μm) were patterned in the format of a 384-well plate. The alginate was then encapsulated inside an inert polymer, PEG-dimethyl ether (PEG-DE, Sigma, #445908, 2 kDa), which has a transition temperature at 53° C. and also dissolves rapidly in water. To do this, the alginate fibers were first air-dried, and then the PEG-DE solution was loaded into the channel to encase the alginate fibers at 70° C. under a vacuum, then solidified at room temperature. The PDMS mold was then removed to leave behind an array of alginate fiber networks encapsulated in PEG-DE on a polystyrene sheet. Finally, the polystyrene sheet was assembled onto the base of a bottomless 384-well plate, encasing and sealing the alginate networks with an inert polyurethane glue (1552-2T50, GS Polymers).

### Example 2: Fabrication of a 3D Cell Culture Device

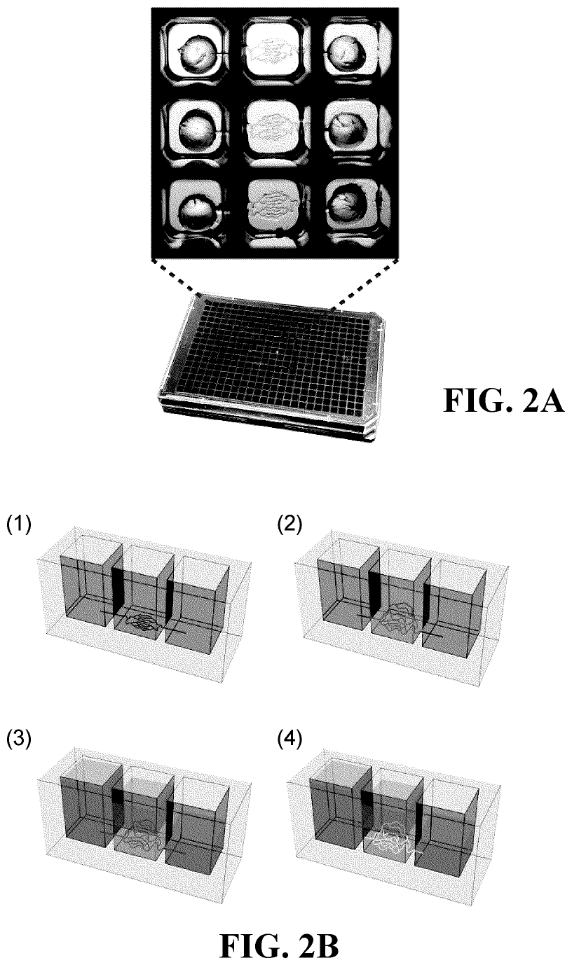

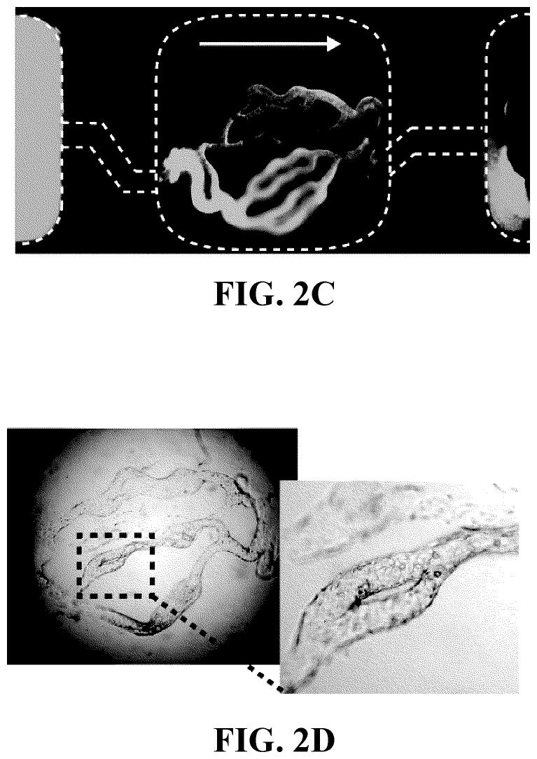

Each well of a 384-well plate made in accordance with the method described in Example 1 was first washed with distilled water to dissolve away the PEG-DE shell and reveal the alginate fibers (FIG. 2B(1)). Next, 20 μL, of a 90:10 v/v mixture of Collagen I and Matrigel™ (354234, Corning), and 5 μL, of PBS, were dispensed onto the alginate fibers and maintained at 4° C. for 30 min to rehydrate the alginate networks (FIG. 2B(2)). During incubation, the dried alginate fibers quickly swelled, detached from the polystyrene base, and dynamically changed shape three-dimensionally inside the hydrogel solution. Next, the hydrogel solution was crosslinked at 37° C. to lock the alginate network in place (FIG. 2B(2)). Finally, 10 mM of ethylenediaminetetraacetic acid (EDTA) was added with culture media at 37° C. for 60 mM to sequester the calcium and dissolve the alginate fibers, resulting in an open perfusable network (FIG. 2B(3), (4)). The plate was washed with fresh culture media prior to cell seeding. A suspension of human umbilical cord vein endothelial cells at a concentration of 1 million cells/mL was applied to both inlets and outlets to deliver the cells into the networks. Endothelial cells were allowed to attach under static condition for at least 1 h. Media perfusion was initiated with gravity-driven flows by simply tilting the plate at a 20° angle on a programmable tilt stage (tilt direction was changed every 15 mM to maintain perfusion).

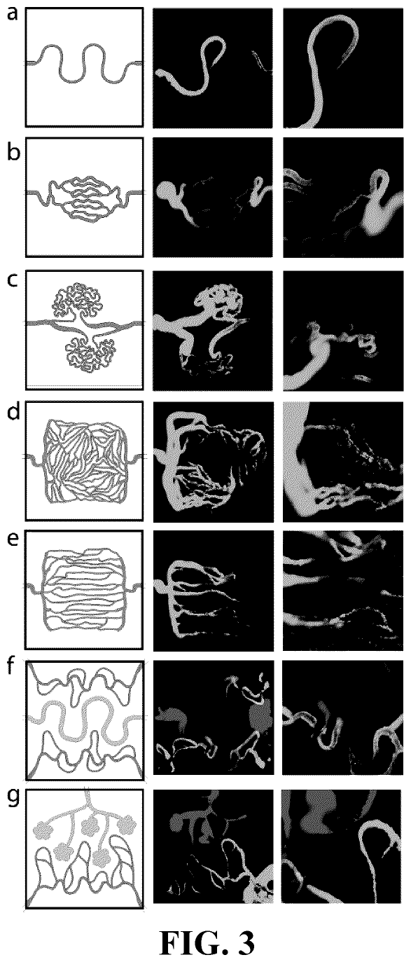

It was found that the alginate networks can detach from the polystyrene base and fold inside the 3D hydrogel (FIGS. 2B-C). The degradation of the alginate fibers resulted in open perfusable networks that span multiple z-planes in 3D (FIG. 2C). Even though the exact 3D positioning of the networks was not pre-determined (this degree of stochasticity in the fabrication of the cell culture device is conductive fidelity), the overall architectural designs (e.g. the diameter, density and shape of the vessels as well as the frequency and location of the branches, etc.) were pre-defined in the initial design (FIG. 3). Hence, distinct organizations of vessel networks originating from various organs or even various parts of an organ can be captured (FIG. 3). For instance, 3D network architectures were formed resembling the convoluted proximal tubules (FIG. 3a) and intricately folded glomerulus vessels in the kidney (FIG. 3c), the densely packed vessels in the liver (FIG. 3d), and the well-aligned vessels in the muscle (FIG. 3e). Further, multiple individually addressable perfusable circuits were incorporated in the same model to reproduce spatially intertwined vascular-tubular networks, such as the proximal tubule and the surrounding microvasculature in the kidney (FIG. 3f) as well as the alveoli and the underlying microvasculature in the lung (FIG. 3g). For example, the tubular network (red, FIG. 3f) can be populated with human primary proximal tubular epithelial cells (H-6015, Cell Biologics) and the branched lobules (red, FIG. 3g) can be populated with human primary alveolar epithelial cells (H-6053, Cell Biologics).

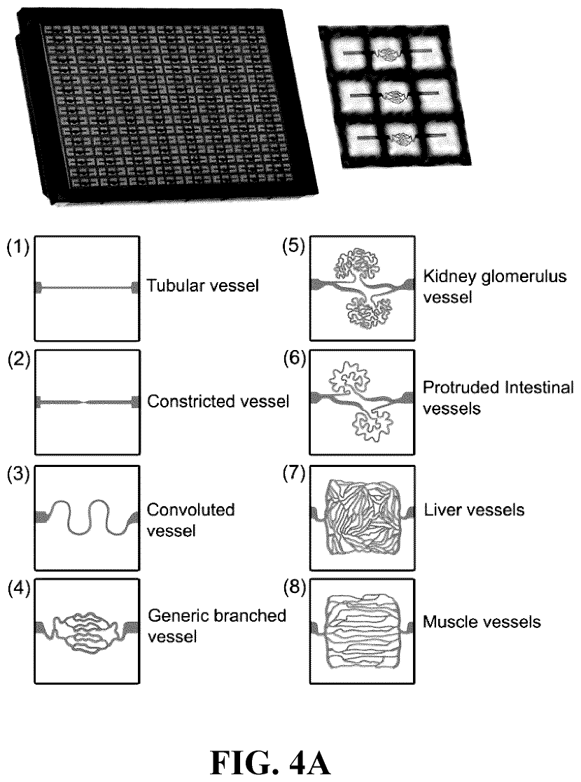

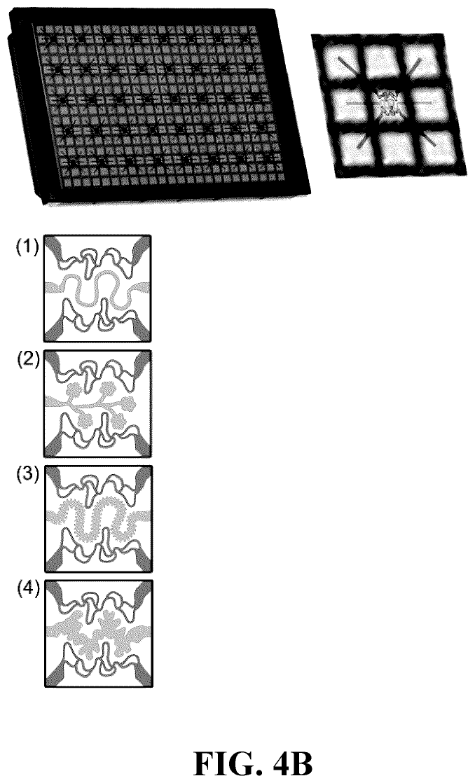

Based on the same manufacturing procedure shown above, a portfolio of plates with 2 different configurations and 12 different designs was developed (FIGS. 4A-B). The first configuration (FIG. 4A) included 128 tissues in a 384-well plate format. Each tissue included a perfusable network with a single inlet and outlet. For this configuration, 8 different designs were developed with increasing complexity to capture the specific characteristics of blood vessel networks found in different organs. A straight tubular vessel design (FIG. 4A(1)) and convoluted vessel design (FIG. 4A(3)) were included to decouple the biological effects of vessel curvature. The straight tubular vessel design will also provide a simple vascular interface that can be easily characterized and modeled. To model vascular disease, a constricted vessel was included (FIG. 4A(2)). Flow dynamics and biological response around the constriction can be visualized and studied. A generic vessel network with bifurcated branching was included to provide a generic vascular bed (FIG. 4A(4)). Four more designs were included to capture the specific architecture of various organ systems (FIG. 4A(5-8)). The second configuration (FIG. 4B) included three independently perfusable networks, each with its own inlet and outlet. The networks labeled in red were seeded with endothelial cells to model vasculature while the network labeled in blue was seeded with various epithelial cells to model the organ-specific tubular structures. Together the vasculature and the tubular networks formed an intercommunicating system that can carry out physiological functions.

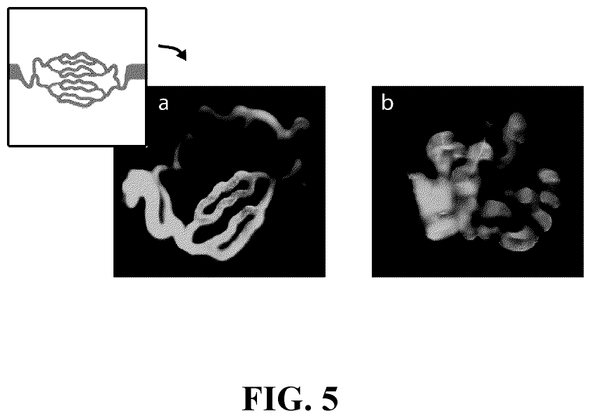

It was also found that the final shape of a 3D perfusable network can be varied depending on the stiffness of the hydrogel formulation used. As shown in FIG. 5, 3D perfusable networks fabricated from the same initial branched-network design but with hydrogel formulations of different stiffness achieved different final shapes. In particular, encapsulation of alginate patterned according to a branched-network design in a softer hydrogel formulation containing 80% (v/v) Matrigel and 20% (v/v) PBS led to formation of a 3D perfusable network suitable for modelling a kidney glomerulus vessel (FIG. 5b), different from the 3D perfusable network formed from a stiffer formulation containing 70% (v/v) collagen, 10% (v/v) Matrigel, and 20% (v/v) PBS (FIG. 5a).

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the scope of the appended claims.

It is to be understood that any numerical value inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used herein, whether in the specification or the appended claims, the transitional terms “comprising”, “including”, “carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the invention disclosed and/or claimed herein.

1. A chamber for cell culture comprising a sacrificial material and a first orifice, wherein the sacrificial material comprises a patterned portion and a first extension portion and dynamically changes shape three-dimensionally upon exposure to a hydrogel solution, and wherein the first extension portion extends to the first orifice and anchors the patterned portion within the chamber.

2. The chamber of claim 1, wherein the first extension portion extends through the first orifice.

3. The chamber of claim 2, wherein the first extension portion at least partially seals the first orifice upon exposure to the hydrogel solution, thereby anchoring the patterned portion.

4. The chamber of claim 1, wherein the chamber further comprises a second orifice and the sacrificial material further comprises a second extension portion, and wherein the second extension portion extends to the second orifice and optionally anchors the patterned portion within the chamber.

5. The chamber of claim 1, wherein the sacrificial material is alginate, gelatin, Matrigel®, agarose, collagen, polyesters, fibrin, or a combination thereof.

6. The chamber of claim 1, wherein the sacrificial material is alginate.

7. The chamber of claim 1, wherein the size of the cross section of the sacrificial material is from about 100 μm² to about 22,500 μm².

8. The chamber of claim 1, wherein the size of the cross section of the sacrificial material is from about 400 μm² to about 10,000 μm².

9. The chamber of claim 1, wherein the patterned portion is in the form of one or more networks.

10. The chamber of claim 9, wherein the network mimics a blood or lymph vessel network, the architecture of an organ or a tissue, or a cavity of an organ or a tissue.

11. The chamber of claim 1, wherein the patterned portion of the sacrificial material is removably attached to the bottom surface of the chamber.

12. The chamber of claim 11, wherein the patterned portion at least partially detaches from the bottom surface of the chamber upon exposure to the hydrogel solution.

13. A cell culture device comprising a first chamber and a second chamber, wherein the first chamber comprises a sacrificial material and a first orifice, wherein the sacrificial material comprises a patterned portion and a first extension portion and dynamically changes shape three-dimensionally upon exposure to a hydrogel solution, wherein the first extension portion extends to the first orifice and anchors the patterned portion within the first chamber, and wherein the second chamber is in fluid communication with the first chamber via the first orifice.

14. The cell culture device of claim 13, wherein the first extension portion extends through the first orifice and into the second chamber.

15. The cell culture device of claim 14, wherein the first extension portion at least partially seals the first orifice upon exposure to the hydrogel solution, thereby anchoring the patterned portion.

16. The cell culture device of claim 13, wherein the sacrificial material is alginate, gelatin, Matrigel®, agarose, collagen, polyesters, fibrin, or a combination thereof.

17. The cell culture device of claim 13, wherein the size of the cross section of the sacrificial material is from about 100 μm² to about 22,500 μm².

18. A method of constructing a chamber for cell culture, comprising the steps of:
a. assembling a mold comprising a template sheet patterned with a network and a backing sheet;
b. casting a sacrificial material in the mold;
c. solidifying the sacrificial material within the patterned network to form a patterned portion and at least one extension portion;
d. removing the template sheet from the sacrificial material and backing sheet; and
e. assembling a bottomless chamber for cell culture onto the backing sheet such that the patterned portion of the sacrificial material is anchored within the chamber, and the extension portion of the sacrificial material extends to an orifice of the chamber.

19. A method of constructing a 3D perfusable network, comprising the steps of:
a. adding a hydrogel solution to the chamber of claim 1, such that the sacrificial material is completely immersed within the hydrogel solution;
b. cross-linking the hydrogel solution; and
c. degrading the sacrificial material.

20. (canceled)

21. A method of constructing a 3D perfusable network, comprising the steps of:
a. adding a hydrogel solution to the cell culture device of claim 13, such that the sacrificial material is completely immersed within the hydrogel solution;
b. cross-linking the hydrogel solution; and
c. degrading the sacrificial material.
